(19) United States
(12) Patent Application Publication
(10) Pub. No.: US 2023/0285326 A1
(43) Pub. Date: Sep. 14, 2023

(54) EPINEPHRINE FORMULATIONS

(71) Applicant: Par Pharmaceutical, Inc., Chestnut Ridge, NY (US)

(72) Inventors: Sunil Maharu PATIL, Mumbai (IN); Atul Arvind BODKHE, Thane (IN); Satish Shamlal PATIL, Thane (IN)

(21) Appl. No.: 17/694,165

(22) Filed: Mar. 14, 2022

(51) Int. Cl. A61K 31/137; A61K 9/00

(52) U.S. Cl. CPC A61K 31/137; A61K 9/0019

(57) ABSTRACT

A method of making a pharmaceutical composition in a pre-filled syringe including preparing a batch solution by providing a solvent having an initial dissolved oxygen content, sparging the solvent with a first inert gas until the solvent has a reduced dissolved oxygen content that is lower than the initial dissolved oxygen content, and combining the solvent with an active agent and an antioxidant. The solvent is continuously sparged with the first inert gas during combination with the active agent and the antioxidant. The method also includes introducing at least a portion of the batch solution into a syringe while sparging the batch solution with a second inert gas that is the same as or different from the first inert gas. The pharmaceutical composition in the pre-filled syringe has a concentration of total impurities of the active agent of no more than about 5.5% (w/v) after three months of storage in accelerated storage conditions. Also described are pharmaceutical compositions in a pre-filled syringe.

### FIELD OF THE INVENTION

The present disclosure is directed to pharmaceutical compositions, methods of administration, and methods of making the same.

### BACKGROUND

Epinephrine, also known as 1, 2-Benzenediol, 4-[(1R)-1-hydroxy-2-(methylamino)ethyl]-, or (−)-3,4-Dihydroxy-α-[2-(methylamino)ethyl]benzyl alcohol, is the active principle of the adrenal medulla and an endogenous catecholamine which acts directly on both alpha and beta adrenergic receptors. When used in pharmaceutical compositions, epinephrine can act as a non-selective alpha and beta adrenergic agonist and can work rapidly to improve breathing, stimulate the heart, raise dropping blood pressure, reverse hives, and reduce swelling of the face, lips, and throat. Uses for epinephrine include emergency treatment of allergic reactions (Type 1), including anaphylaxis, induction and maintenance of mydriasis during intraocular surgery, treatment of bronchospasm, sensitivity reactions, cardiac arrhythmias, GI and renal hemorrhage, superficial bleeding, premature labor, hypoglycemia, and cardiogenic, hemorrhagic, and traumatic shock. Epinephrine can also be used to increase blood flow in Advanced Cardiovascular Life Support during CPR, as an adjunct to local anesthesia, and for radiographic uses.

One example of a pharmaceutical composition with epinephrine as an active agent is Adrenalin®, a sympathomimetic agent manufactured and distributed by Par Sterile Products, LLC. Adrenalin® is an epinephrine injection suitable for subcutaneous, intracameral, and intramuscular administration. When diluted, it may also be administered as ophthalmic irrigation or intracameral injection. Adrenalin® is used primarily as an emergency treatment of allergic reactions (Type 1), including anaphylaxis, and for induction and maintenance of mydriasis during intraocular surgery.

Adrenalin® is a clear, colorless solution containing 1 mg/mL epinephrine in a clear glass vial. Adrenalin® is currently available in 1 mL single-dose and 30 mL multi-dose formulations. For the 1 mL product, each 1 mL of Adrenalin® contains 1 mg epinephrine, 9.0 mg sodium chloride, 1.0 mg sodium metabisulfite, hydrochloric acid to adjust pH, and water for injection. For the 30 mL product, each 1 mL of Adrenalin® solution contains 1 mg epinephrine, 9.0 mg sodium chloride, 1.5 mg sodium metabisulfite, hydrochloric acid to adjust pH, 5.4 mg chlorobutanol as a preservative and water for injection. The pH range of Adrenalin® is 2.2-5.0.

Adrenalin® 1 mL is FDA approved for an 18 month shelf life. The Adrenalin® 30 mL product is approved for an even shorter shelf life of 14 months. Shelf life is limited by the formation of degradants, which mainly comprise epinephrine sulfonic acid (ESA) and D-epinephrine, an enantiomer of L-epinephrine that has insignificant therapeutic activity. The currently approved Adrenalin® 1 mL product has a total impurity limit of <20%. Adrenalin® 1 mL has a concentration limit of <13.5% ESA and <9.5% D-epinephrine. The currently approved Adrenalin® 30 mL product has a total impurity limit of <20%. Adrenalin®30 mL has a concentration limit of <14.5% ESA and <9.5% D-epinephrine.

There is currently a need in the art for improved epinephrine-containing pharmaceuticals. It is an object of the present invention to provide an epinephrine-containing pharmaceutical composition that addresses some of the limitations of present formulations.

### SUMMARY OF THE INVENTION

The present disclosure is directed to a pharmaceutical composition and methods of making the same. The method may comprise preparing a batch solution, wherein preparing the batch solution comprises providing a solvent having an initial dissolved oxygen content, sparging the solvent with a first inert gas until the solvent has a reduced dissolved oxygen content that is lower than the initial dissolved oxygen content, and combining the solvent with one or more components of the composition, wherein the solvent is continuously sparged with the first inert gas during combination with at least one of the one or more components. The method may further comprise introducing at least a portion of the batch solution into a syringe to provide the pharmaceutical composition in a pre-filled syringe, wherein the batch solution is sparged with a second inert gas when provided in the pre-filled syringe.

### BRIEF DESCRIPTION OF THE FIGURES

FIG. 1 is a chromatogram of the Impurities Marker Solution for an epinephrine composition according to the invention using isocratic HILIC with 15% water.

FIG. 2 is a chromatogram of the Impurities Marker Solution for an epinephrine composition according to the invention using isocratic HILIC with 18% water.

FIG. 3 is a chromatogram of the Impurities Marker Solution for an epinephrine composition according to the invention using isocratic HILIC with 22% water.

FIG. 4 is a chromatogram of the Impurities Marker Solution for an epinephrine composition according to the invention using gradient HILIC separation.

### DETAILED DESCRIPTION OF THE INVENTION

The present disclosure is directed to a pharmaceutical composition and methods of making and using the same. According to some aspects, the composition may comprise at least one of an active agent, an antioxidant, a pH adjusting agent, a tonicity adjusting agent, a chelating agent, a buffer system, and a solvent. The composition may be useful for emergency treatment of allergic reactions (Type 1), including anaphylaxis, induction and maintenance of mydriasis during intraocular surgery, treatment of bronchospasm, sensitivity reactions, cardiac arrhythmias, GI and renal hemorrhage, superficial bleeding, premature labor, hypoglycemia, and cardiogenic, hemorrhagic, and traumatic shock. The composition may also be used to increase blood flow in ACLS during CPR, as an adjunct to local anesthesia, and for radiographic uses. The composition may be administrable via subcutaneous, intracameral, intravenous, and intramuscular injection, infusion, intra-arterial administration, intracardiac injection, endotracheal administration, intraosseous administration, oral inhalation, topical administration, and as ophthalmic irrigation.

According to some aspects, the composition may be provided in a container, such as a pre-filled syringe. The composition may have an acceptable initial impurity level and/or an acceptable impurity level after a certain period of shelf life.

The present disclosure is also directed to a method of making a composition as described herein. The method may comprise preparing a batch solution, wherein preparing the batch solution comprises providing a solvent having an initial dissolved oxygen content, sparging the solvent with a first inert gas until the solvent has a reduced dissolved oxygen content that is lower than the initial dissolved oxygen content, and combining the solvent with one or more components of the composition, wherein the solvent is continuously sparged with the first inert gas during combination with at least one of the one or more components. The method may further comprise introducing at least a portion of the batch solution into a syringe to provide the pharmaceutical composition in the pre-filled syringe, wherein the batch solution is sparged with a second inert gas when provided in the pre-filled syringe. The second inert gas may be different from or the same as the first inert gas.

As used herein, the term “about” means ±5% and optionally ±1% of the provided value.

According to some aspects, the active agent may comprise epinephrine and/or salts thereof. Examples of epinephrine salts include, but are not limited to, acetate, carbonate, citrate, hydrochloride, hydrocyanide, hydrofluoride, nitrate, nitrite, phosphate, tartrate, and sulfate salts.

According to some aspects, the active agent may be provided in the composition at a concentration sufficient for any of the uses described herein. In some non-limiting examples, the active agent is provided at a concentration of from about 0.01 to about 2 mg/mL, optionally of from about 0.01 to about 1 mg/mL, optionally of from about 0.01 to about 0.5 mg/mL, optionally of from about 0.01 to about 0.3 mg/mL, optionally of from about 0.01 to about 0.2 mg/mL, and optionally about 0.1 mg/mL.

According to some aspects, the composition may comprise at least one antioxidant. As used herein, the term “antioxidant” refers to a component in a composition that may at least in part prevent and/or inhibit the formation of unacceptable amounts of oxidative degradants in the composition after a certain period of shelf life. For example, the antioxidant may react with oxygen that might otherwise compromise the composition by producing impurities in the composition. Non-limiting examples of oxidative degradants of epinephrine include adrenalone, adrenochrome, adrenolutin, adrenochrome sulfonate, melanins, D-epinephrine, and epinephrine sulfonic acid (ESA).

Example antioxidants include, but are not limited to, sulfites such as sodium bisulfite, sodium metabisulfite, and combinations thereof.

According to some aspects, the antioxidant may be provided in the composition at a concentration of from about 0.001 to about 0.2 mg/mL, optionally from about 0.001 to about 0.1 mg/mL, optionally from about 0.01 to about 0.09 mg/mL, optionally from about 0.02 to about 0.08 mg/mL, optionally from about 0.03 to about 0.07 mg/mL, optionally from about 0.04 to about 0.06 mg/mL, and optionally about 0.0457 mg/mL.

According to some aspects, the composition may comprise at least one tonicity adjusting agent. The term “tonicity” refers to the effective osmotic pressure equivalent of a solution or composition. According to some aspects, the tonicity adjusting agent may be present in the composition at a concentration sufficient to maintain the tonicity of the composition in a physiologically acceptable range.

Example tonicity adjusting agents include, but are not limited to, glucose, glycerin, hydroxypropyl methyl cellulose, mannitol, polysorbate, propylene glycol, sodium bromide, sodium chloride, sodium iodide, sorbitol, urea, xylitol, and combinations thereof.

According to some aspects, the tonicity adjusting agent may be provided in the composition at a concentration of from about 0.1 to about 20 mg/mL, optionally of from about 3 to about 15 mg/mL, optionally of from about 4 to about 14 mg/mL, optionally of from about 5 to about 13 mg/mL, optionally of from about 6 to about 12 mg/mL, optionally of from about 7 to about 11 mg/mL, optionally of from about 8 to about 10 mg/mL, and optionally about 9 mg/mL.

According to some aspects, the composition may comprise at least one chelating agent. The term “chelating agent” refers to a substance capable of chelation, i.e., the formation or presence of two or more separate coordinate bonds between a polydentate ligand and a single central atom. The chelating agent may be provided at a concentration sufficient to reduce the catalytic activity of trace metals present in the composition. For example, the chelating agent may chelate trace metals in the composition that may otherwise increase and/or accelerate the degradation of components in the composition. Examples of trace metals include, but are not limited to, iron, magnesium, lithium, zinc, copper, chromium, nickel, cobalt, vanadium, arsenic, molybdenum, manganese, selenium, and calcium.

Examples of chelating agents include, but are not limited to, TPA (Tris[(2-pyridyl)methyl]amine), phanquinone (4,7-phenanthroline-5,6-dione), clioquinol (PN Gerolymatos SA), chloroquinol, penicillamine, trientine, N,N′-diethyldithiocarbamate (DDC), 2,3,2′-tetraamine (2,3,2′-tet), neocuproine, N,N,N′,N′-tetrakis(2-pyridylmethyl) ethylenediamine (TPEN), 1,10-phenanthroline (PHE), tetraethylenepentamine (TEPA), triethylene tetraamine and tris(2-carboxyethyl)phosphine (TCEP), bathophenanthroline disulfonic acid (BPADA), Disodium Edetate Dihydrate (EDTA), ethylene glycol (bis) aminoethyl ether tetra acetic acid (EGTA), nitrilotriacetic acid, TIRON™, N,N-bis(2-hydroxyethyl)glycine (bicine), O,O′-bis(2-aminophenyl ethylene glycol) ethylenediamine-N,N,N′,N′-tetraacetic acid (BAPTA), trans-1,2-diamino cyclohexane-ethylenediamine-N,N,N′,N′-tetraacetic acid (CyDTA), 1,3-diamino-2-hydroxy-propane-ethylenediamine-N,N,N′,N′-tetraacetic acid (DPTA-OH), ethylene-diamine-N,N′-dipropionic acid dihydrochloride (EDDP), ethylenediamine-N,N′-bis(methylenephosphonic acid) hemihydrate (EDDPO), ethylenediamine-N,N,N′,N′-tetrakis(methylenephosphonic acid) (EDTPO), N,N′-bis(2-hydroxybenzyl)ethylene diamine-N,N′-diacetic acid (HBED), 1,6-hexamethylenediamine-N,N,N′,N′-tetraacetic acid (HDTA, or HEDTA), N-(2-hydroxyethyl)iminodiacetic acid (HIDA), iminodiacetic acid (IDA), 1,2-diaminopropane-N,N,N′,N′-tetraacetic acid (methyl-EDTA), nitriltriacetic acid (NTA), nitrilotripropionic acid (NTP), nitrilotris (methylenephosphonic acid) trisodium salt (NTPO), triethylenetetramine-N,N,N′,N″,N″-hexaacetic acid (TTHA), bathocuproine, bathophenanthroline, TETA, citric acid, salicylic acid, and/or malic acid, including analogues, derivatives, and pharmaceutically acceptable salts thereof.

According to some aspects, the composition may comprise a pH adjusting agent. The term “pH adjusting agent” refers to a component of a composition that modifies the composition's pH. The pH adjusting agent may be present in the composition such that the composition's pH is in a pharmaceutically acceptable range (i.e., not toxic or producing unacceptable side effects).

According to some aspects, the pH adjusting agent may be provided in the composition at a concentration of from about 0.01 to about 2.0 mg/mL, optionally from about 0.01 to about 1.0 mg/mL, optionally from about 0.1 to about 0.9 mg/mL, optionally from about 0.2 to about 0.9 mg/mL, optionally from about 0.3 to about 0.8 mg/mL, optionally from about 0.4 to about 0.8 mg/mL, optionally from about 0.5 to about 0.7 mg/mL, and optionally about 0.606 mg/mL.

According to some aspects, the pH adjusting agent comprises one or more of acetic acid, adipic acid, ascorbic acid, citric acid, hydrochloric acid, lactic acid, malic acid, monopotassium phosphate, monosodium phosphate, phosphoric acid, pyrophosphoric acid, succinic acid, sulfuric acid, tartaric acid, and solutions thereof. According to some aspects, the pH adjusting agent may be provided as a solution, for example, a 4% solution in water for injection (WFI).

According to some aspects, the composition may comprise a buffer system having one or more buffer components. The term “buffer system” refers to a component of a composition that provides a resistance to significant change in pH caused by a strong acid or base. A buffer system may comprise two or more buffer components, such as a weak acid and its conjugate base. A buffer system may provide a resistance to a significant pH change by interacting with a strong acid or strong base in a composition or solution, thereby at least partially preventing the pH of the composition or solution from changing significantly.

Generally, a buffer system has one or more buffer ranges wherein the buffer system has the ability to provide resistance to significant pH change. When a composition or solution comprising the buffer system has a pH inside the buffer system's buffer range, the pH of the composition or solution will not change significantly with the addition of equimolar amounts of a strong acid or strong base.

The buffer range of a buffer system is related to the acid dissociation constant (Kₐ) of one or more weak acids comprised by the buffer system. The term “acid dissociation constant” refers to the equilibrium constant of a dissociation reaction of an acid. The midpoint of a buffer range for a buffer system is generally about the logarithmic measure of the acid dissociation constant (i.e., the pKₐ, equal to −log₁₀Kₐ) of a weak acid comprised by the buffer system.

According to some aspects, the buffer system may comprise one or more buffer components having one or more pKₐ values. The one or more buffer components may comprise a pKₐ value that is within +/−1 unit of the initial pH of the pharmaceutical composition in the form of a solution without the pH raising agent. According to some aspects, the one or more buffer components may have a pKₐ value within the range of about 2 to 5, optionally within the range of about 3 to 4.5, optionally within the range of about 3.5 to 4.5, optionally about 4, and optionally about 4.11. According to some aspects, the one or more buffer components may have a buffer range from a pH of about 2 to 5, optionally from a pH of about 3 to 4.5, and optionally from a pH of about 3.5 to 4.5.

The one or more buffer components may provide a resistance to a significant change in pH that other components of the composition may otherwise cause in the absence of the one or more buffer components. For example, the one or more buffer components may provide a resistance to the composition's pH being significantly lowered by degradants and/or reaction products of other components of the composition. According to some aspects, the one or more buffer components may help resist an increase in acidity associated with the chemical reaction of an antioxidant.

According to some aspects, each of the one or more buffer components may be present in the composition at a concentration in the range of about 0.01 and 0.4 mg/mL, optionally in the range of about 0.1 and 0.3 mg/mL, optionally in the range of about 0.15 and 0.25 mg/mL, and optionally about 0.225 mg/mL. According to some aspects, each of the one or more buffer components may be present in the composition at a concentration in the range of about 0.01 and 2.0 mg/mL, optionally in the range of about 0.01 and 1.0 mg/mL, optionally in the range of about 0.01 and 0.5 mg/mL, optionally in the range of about 0.01 and 0.2 mg/mL, and optionally about 0.1 mg/mL.

The one or more buffer components may be present at a concentration that provides a resistance to a significant change in the composition's pH despite physical and chemical changes of the composition after a certain period of shelf life.

According to some aspects, the one or more buffer components are selected such that the degradation of other components of the composition is slowed or reduced, as compared to a composition in which the one or more buffer components are not present. For example, the one or more buffer components may at least in part help prevent significant degradation of an antioxidant after a certain period of shelf life.

According to some aspects, the one or more buffer components and concentration(s) thereof are such that compositions conform to the limit on total acidity imposed by the United States Pharmacopeia (USP) 37-NF 32, S2, monograph, effective from Dec. 1, 2014. For example, the one or more buffer components may be selected such that when 5.0 mL of the composition is titrated with 0.01 N NaOH, the total amount of titrant necessary to reach a pH of 7.4 is no more than 25 mL. The one or more buffer components may be selected such that the composition has a titratable acid concentration of in the range of about 30 to 70 mM, preferably in the range of about 40 and 60 mM, and most preferably about 50 mM.

The one or more buffer components may be suitable for subcutaneous, intracameral, intramuscular, parenteral, and/or ophthalmic use.

According to some aspects, the one or more buffer components may comprise the acids or salt forms of one or more of lactate, tartarate, glutamate, malate, citrate, gluconate, benzoate, succinate, acetate, glycine, and aspartate. According to some aspects, the one or more buffer components may comprise lithium hydroxide, sodium hydroxide, potassium hydroxide, rubidium hydroxide, cesium hydroxide, calcium hydroxide, strontium hydroxide, and/or barium hydroxide. According to some aspects, the buffer system may comprise tartaric acid and sodium hydroxide.

The composition may comprise a solvent. According to some aspects, the solvent may be acceptable for pharmaceutical administration. Examples of methods of pharmaceutical administration include, but are not limited to, subcutaneous, intracameral, intravenous, and intramuscular injection, infusion, intra-arterial administration, intracardiac injection, endotracheal administration, intraosseous administration, oral inhalation, topical administration, and as ophthalmic irrigation.

According to some aspects, the solvent may comprise one or more of acetic acid, acetone, acetonitrile, animal oil, aqueous buffer, benzene, bisabolol, butanol, carbon tetrachloride, chlorobenzene, chloroform, dimethylformamide, dioxane, essential oil, ethanol, ethyl acetate, ethyl oleate, ethylene chloride, fatty acid esters, glycerin, glycofurol, hexane, hydrogenated vegetable oil, isopropanol, isopropyl alcohol, isopropyl myristate, isopropyl palmitate, methanol, methylene chloride, mineral oil, polyethylene glycol, polyol, propylene glycol, silicone fluid glyceride, squalane, terpene, tetrahydrofuran, toluene, triacetin, tributyl citrate, triethyl citrate, vegetable oil, and water. In one example, the solvent comprises water for injection (WFI). As used herein, the term “water for injection” or “WFI” refers to sterile, non-pyrogenic, distilled water suitable for intravenous administration after addition of a suitable solute. According to some aspects, WFI may refer to water that meets USP requirements for WFI. For example, WFI may refer to water that comprises less than 0.25 USP Endotoxin Unit/mL per the Bacterial Endotoxins Test <85>, meets the requirements as set forth in Water Conductivity, Bulk Water <645>, and meets the requirements as set forth in Total Organic Carbon <643>.

The solvent may be present in an amount that brings the composition to a final volume which is suitable for administration. For example, the final volume may be about 1 mL. In another example, the final volume may be about 10 mL, optionally about 10.2 mL. In another example, the final volume may be about 30 mL.

The composition according to the present disclosure may have an acceptable initial impurity level and an acceptable impurity level after a certain period of shelf life. As used herein, the term “impurity” refers to an undesired substance in a composition. It should be understood that one or more impurities may be present in an initial composition and/or may be formed after a certain period of shelf life of a composition. For example, one or more impurities may be formed via degradation of one or more components of the composition, such as the active agent. Sources of degradation include, but are not limited to, oxidation, racemization, visible light, ultraviolet light, moisture, heat (including heat from a sterilization process), changes in pH, and composition component interactions. Example impurities for epinephrine include, but are not limited to, D-epinephrine, ESA, Impurity A, Impurity B, Unknown C, adrenalone, adrenochrome sulfonate, adrenochrome, adrenolutin, melanins, and analogs thereof.

According to some aspects, the composition may have no more than about 20% of total impurities after a certain period of shelf life, more preferably no more than about 19.5%, more preferably no more than about 19%, more preferably no more than about 18.5%, preferably no more than about 18%, more preferably no more than about 17.5%, more preferably no more than about 17%, more preferably no more than about 16.5%, more preferably no more than about 16%, more preferably no more than about 15.5%, more preferably no more than about 15%, more preferably no more than about 14.5%, more preferably no more than about 14%, more preferably no more than about 13.5%, more preferably no more than about 13%, more preferably no more than about 12.5%, more preferably no more than about 12%, more preferably no more than about 11.5%, more preferably no more than about 11%, more preferably no more than about 10.5%, more preferably no more than about 10%, more preferably no more than about 9.5%, more preferably no more than about 9%, more preferably no more than about 8.5%, more preferably no more than about 8%, more preferably no more than about 7.5%, more preferably no more than about 7%, more preferably no more than about 6.5%, more preferably no more than about 6%, more preferably no more than about 5.5%, and most preferably no more than about 5%.

According to some aspects, the composition may have no more than about 20% of total impurities after a certain period of shelf life, preferably no more than about 19.9%, more preferably no more than about 19.8%, more preferably no more than about 19.7%, more preferably no more than about 19.6%, more preferably no more than about 19.5%, more preferably no more than about 19.4%, more preferably no more than about 19.3%, more preferably no more than about 19.2%, more preferably no more than about 19.1%, more preferably no more than about 19%, more preferably no more than about 18.9%, more preferably no more than about 18.8%, more preferably no more than about 18.7%, more preferably no more than about 18.6%, more preferably no more than about 18.5%, more preferably no more than about 18.4%, more preferably no more than about 18.3%, more preferably no more than about 18.2%, more preferably no more than about 18.1%, more preferably no more than about 18%, more preferably no more than about 17.9%, more preferably no more than about 17.8%, more preferably no more than about 17.7%, more preferably no more than about 17.6%, more preferably no more than about 17.5%, more preferably no more than about 17.4%, more preferably no more than about 17.3%, more preferably no more than about 17.2%, more preferably no more than about 17.1%, more preferably no more than about 17%, more preferably no more than about 16.9%, more preferably no more than about 16.8%, more preferably no more than about 16.7%, more preferably no more than about 16.6%, more preferably no more than about 16.5%, more preferably no more than about 16.4%, more preferably no more than about 16.3%, more preferably no more than about 16.2%, more preferably no more than about 16.1%, more preferably no more than about 16%, more preferably no more than about 15.9%, more preferably no more than about 15.8%, more preferably no more than about 15.7%, more preferably no more than about 15.6%, more preferably no more than about 15.5%, more preferably no more than about 15.4%, more preferably no more than about 15.3%, more preferably no more than about 15.2%, more preferably no more than about 15.1%, more preferably no more than about 15%, more preferably no more than about 14.9%, more preferably no more than about 14.8%, more preferably no more than about 14.7%, more preferably no more than about 14.6%, more preferably no more than about 14.5%, more preferably no more than about 14.4%, more preferably no more than about 14.3%, more preferably no more than about 14.2%, more preferably no more than about 14.1%, more preferably no more than about 14%, more preferably no more than about 13.9%, more preferably no more than about 13.8%, more preferably no more than about 13.7%, more preferably no more than about 13.6%, more preferably no more than about 13.5%, more preferably no more than about 13.4%, more preferably no more than about 13.3%, more preferably no more than about 13.2%, more preferably no more than about 13.1%, more preferably no more than about 13%, more preferably no more than about 12.9%, more preferably no more than about 12.8%, more preferably no more than about 12.7%, more preferably no more than about 12.6%, more preferably no more than about 12.5%, more preferably no more than about 12.4%, more preferably no more than about 12.3%, more preferably no more than about 12.2%, more preferably no more than about 12.1%, more preferably no more than about 12%, more preferably no more than about 11.9%, more preferably no more than about 11.8%, more preferably no more than about 11.7%, more preferably no more than about 11.6%, more preferably no more than about 11.5%, more preferably no more than about 11.4%, more preferably no more than about 11.3%, more preferably no more than about 11.2%, more preferably no more than about 11.1%, more preferably no more than about 11%, more preferably no more than about 10.9%, more preferably no more than about 10.8%, more preferably no more than about 10.7%, more preferably no more than about 10.6%, more preferably no more than about 10.5%, more preferably no more than about 10.4%, more preferably no more than about 10.3%, more preferably no more than about 10.2%, more preferably no more than about 10.1%, more preferably no more than about 10%, more preferably no more than about 9.9%, more preferably no more than about 9.8%, more preferably no more than about 9.7%, more preferably no more than about 9.6%, more preferably no more than about 9.5%, more preferably no more than about 9.4%, more preferably no more than about 9.3%, more preferably no more than about 9.2%, more preferably no more than about 9.1%, more preferably no more than about 9%, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

According to some aspects, the concentration of impurities present in the composition after a certain period of shelf life may be attributed at least partially to degradation of components of the composition. According to some aspects, the concentration of impurities present in the composition at the end of shelf life may be attributed at least partially to epinephrine degradation. Epinephrine degradation may be a result of physical or chemical stress. Examples of stresses include, but are not limited to, oxygen, pH, bisulfite, light, process surface compatibility, and soluble trace metals.

According to some aspects, the concentration of D-epinephrine in the composition after a certain period of shelf life may be no more than about 9.5%, preferably no more than about 9%, more preferably no more than about 8.5%, more preferably no more than about 8%, more preferably no more than about 7.5%, more preferably no more than about 7%, more preferably no more than about 6.5%, more preferably no more than about 6%, more preferably no more than about 5.5%, more preferably no more than about 5%, more preferably no more than about 4.5%, more preferably no more than about 4%, more preferably no more than about 3.5%, more preferably no more than than about 3%, more preferably no more than about 2.5%, more preferably no more than about 2%, more preferably no more than about 1.5%, more preferably no more than about 1%, and most preferably no more than about 0.5%.

According to some aspects, the concentration of D-epinephrine in the composition after a period of shelf life may be no more than about 9.5%, preferably no more than about 9.4%, more preferably no more than about 9.3%, more preferably no more than about 9.2%, more preferably no more than about 9.1%, more preferably no more than about 9%, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to some aspects, the initial concentration of D-epinephrine in the composition may be no more than about 2.5%, preferably no more than about 2%, more preferably no more than about 1.5%, more preferably no more than about 1%, and most preferably no more than about 0.5%.

According to some aspects, the composition may have a concentration of ESA after a certain period of shelf life of no more than about 14.5%, preferably no more than about 14%, more preferably no more than about 13.5%, more preferably no more than about 13%, more preferably no more than about 12.5%, more preferably no more than about 12%, more preferably no more than about 11.5%, more preferably no more than about 11%, more preferably no more than about 10.5%, more preferably no more than about 10%, more preferably no more than about 9.5%, more preferably no more than about 9%, more preferably no more than about 8.5%, more preferably no more than about 8%, more preferably no more than about 7.5%, more preferably no more than about 7%, more preferably no more than about 6.5%, more preferably no more than about 6%, and most preferably no more than about 5.5%.

According to some aspects, the concentration of ESA in the composition after a certain period of shelf life may be no more than about 14.5%, preferably no more than about 14.4%, more preferably no more than about 14.3%, more preferably no more than about 14.2%, more preferably no more than about 14.1%, more preferably no more than about 14%, more preferably no more than about 13.9%, more preferably no more than about 13.8%, more preferably no more than about 13.7%, more preferably no more than about 13.6%, more preferably no more than about 13.5%, more preferably no more than about 13.4%, more preferably no more than about 13.3%, more preferably no more than about 13.2%, more preferably no more than about 13.1%, more preferably no more than about 13%, more preferably no more than about 12.9%, more preferably no more than about 12.8%, more preferably no more than about 12.7%, more preferably no more than about 12.6%, more preferably no more than about 12.5%, more preferably no more than about 12.4%, more preferably no more than about 12.3%, more preferably no more than about 12.2%, more preferably no more than about 12.1%, more preferably no more than about 12%, more preferably no more than about 11.9%, more preferably no more than about 11.8%, more preferably no more than about 11.7%, more preferably no more than about 11.6%, more preferably no more than about 11.5%, more preferably no more than about 11.4%, more preferably no more than about 11.3%, more preferably no more than about 11.2%, more preferably no more than about 11.1%, more preferably no more than about 11%, more preferably no more than about 10.9%, more preferably no more than about 10.8%, more preferably no more than about 10.7%, more preferably no more than about 10.6%, more preferably no more than about 10.5%, more preferably no more than about 10.4%, more preferably no more than about 10.3%, more preferably no more than about 10.2%, more preferably no more than about 10.1%, more preferably no more than about 10%, more preferably no more than about 9.9%, more preferably no more than about 9.8%, more preferably no more than about 9.7%, more preferably no more than about 9.6%, more preferably no more than about 9.5%, more preferably no more than about 9.4%, more preferably no more than about 9.3%, more preferably no more than about 9.2%, more preferably no more than about 9.1%, more preferably no more than about 9%, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5.0%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4.0%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3.0%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2.0%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1.0%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

According to some aspects, the initial concentration of ESA in the composition may be no more than about 0.5%, preferably no more than about 0.2%, more preferably no more than about 0.1%, more preferably no more than about 0.05%, and most preferably no more than about 0.025%.

According to some aspects, the composition has a low level of oxidative degradants. The term “oxidative degradant” refers to any impurity that may be at least partially attributed to an oxidation reaction involving one or more components of the composition. In some non-limiting examples, the oxidative degradants may be formed via oxidation of epinephrine. Examples of oxidative degradants include, but are not limited to, adrenalone, adrenochrome sulfonate, adrenochrome, adrenolutin, melanins, and analogs thereof.

According to some aspects, the concentration of one of the oxidative degradants in the composition after a certain period of shelf life may be no more than about 1%, preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.05%, more preferably no more than about 0.04%, more preferably no more than about 0.03%, more preferably no more than about 0.02%, and most preferably no more than about 0.01%, after a certain period of shelf life.

According to some aspects, the concentration of more than one or all of the oxidative degradants in the composition after a certain period of shelf life may be no more than about 1%, preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.05%, more preferably no more than about 0.04%, more preferably no more than about 0.03%, more preferably no more than about 0.02%, and most preferably no more than about 0.01%, after a certain period of shelf life.

According to some aspects, the concentration of oxidative degradants present in the composition after a certain period of shelf life may be such that the composition does not undergo a physical change. Examples of physical change include, but are not limited to, color change and insoluble particle formation.

According to some aspects, the composition may have low levels of degradants Impurity A, Impurity B, and/or Unknown C.

Impurity A may be a compound with the following structure:

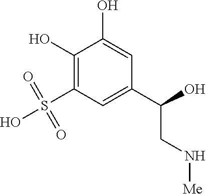 [SMILES: CNC[C@H](O)c1cc(O)c(O)c(S(=O)(=O)O)c1]

Impurity B may be a compound with the following structure:

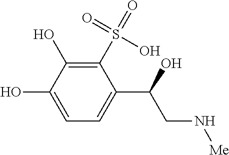 [SMILES: CNC[C@H](O)c1ccc(O)c(O)c1S(=O)(=O)O]

Unknown C may be characterized by a Amax of about 380 nm.

Unknown C may be characterized by its elution peak using HILIC (Hydrophilic Interaction Liquid Chromatography), which differs from one substance to another.

Unknown C may be characterized using an isocratic HILIC separation of mixtures according to the conditions described in Tables 1-3.

Table 1 lists the compositions of the mobile phases that may be used for isocratic HILIC separations.

TABLE 1
Composition of Isocratic Mobile Phases

| | |
|---|---|
| Mobile Phase A | 100 mL Ammonium Formate Buffer |
| | 900 mL Acetonitrile |
| Mobile Phase B | 100 mL Ammonium Formate Buffer |
| | 400 mL Water |
| | 500 mL Acetonitrile |

The Ammonium Formate Buffer of Table 1 may be prepared as follows: 1.5 g of Sodium Chloride, 5.0 mL of Formic Acid, and 3.0 mL of 6N Ammonium Hydroxide is added to 1.0 L of water. The solution may be vacuum filtered through a 0.45 μm nylon membrane. The preparation may be scaled up where necessary.

Table 2 lists different conditions that may be used for isocratic HILIC separations.

TABLE 2
Isocratic Mobile Phases for HILIC Separation

| Isocratic | Mobile | Mobile |
|---|---|---|
| Water % | Phase A % | Phase B % |
| 15 | 87.5 | 12.5 |
| 18 | 80 | 20 |
| 22 | 70 | 30 |

An Epinephrine HILIC Impurities Marker Solution (EpiHILIC IMS) may be prepared by first preparing an Epinephrine/Degradant Bulk Solution as follows: In a clean, clear, 2 L glass bottle, dissolving sodium chloride (6.147 g) and tartaric acid (2.251 g) in about 800 mL of water, adding sodium hydroxide 5.0N (6.25 mL) to the solution and mixing, adding sodium metabisulfite (1.425 g) to the solution and mixing to dissolve, adding epinephrine (1.1334 g) as a solution in HCl 1.0N (6.6 mL) to the solution, and then adding water to a final solution weight of 1005.9, and mixing thoroughly. The Epinephrine/Degradant Bulk Solution may then be allowed to sit in a capped bottle in the hood, under fluorescent light with a light exposure at about 1000 lux, for 12 days with lights continuously on.

The Epinephrine HILIC Impurities Marker Solution (EpiHILIC IMS) may then be prepared in a 500 mL clear glass bottle as follows: adding Epinephrine/Degradant Bulk Solution (500 mL), adding HCl 1.0 N (6.0 mL) and then mixing (with a final pH at 3.2), adding ESA (9.5 mg) and mixing to dissolve, adding adrenochrome (5.6 mg) and mixing to dissolve, preparing adrenalone stock mixture by withdrawing 10 mL of the solution then adding and dissolving adrenalone HCl (4.7 mg), adding adrenalone stock mixture (0.8 mL) back into the bulk mixture, and mixing thoroughly.

An Epinephrine HILIC Impurities Marker Solution (EpiHILIC IMS) which comprises Unknown C may be analyzed using the conditions listed in Table 3.

TABLE 3
Chromatographic Conditions for Isocratic HILIC Separation

| | |
|---|---|
| Column | SeQuant ZIC HILIC PEEK, 150 × 4.6 mm, |
| | 3.5 μm particle size, 100 Å pore |
| | size |
| Flow Rate | 1 mL/min |
| Injection Volume | 25 μL |
| Column Temperature | 35° C. |
| Sample preparation | Dilute exactly 1 volume of sample |
| | (nominal strength of 1 mg/mL |
| | epinephrine) with exactly 3 volumes of |
| | acetonitrile. |
| Detector | Signal-280 nm, Bandwidth- 10 nm; |
| | Reference-Off |
| | Signal-346 nm, Bandwidth- 10 nm; |
| | Reference-Off |
| | Signal-380 nm, Bandwidth- 10 nm; |
| | Reference-Off |
| | Signal-292 nm, Bandwidth-10 nm; |
| | Reference-Off |
| Run Time | 35 minutes |
| Data Acquisition | Integrate impurities between 0 and 30 |
| | minutes |

Tables 4 lists the relevant characteristic peaks of isocratic HILIC separation according to the above specifications at a detection wavelength of 346 nm using the 15% isocratic water preparation according to Table 2.

TABLE 4
Chromatography of the Impurities Marker Solution using
isocratic HILIC with 15% water and 346 nM Detection

| | Time (Minutes) | Absorbance (AU) |
|---|---|---|
| Unknown C | approx. 21 | approx. 0.001 |

Tables 5 lists the relevant characteristic peaks of isocratic HILIC separation according to the above specifications at a detection wavelength of 346 nm using the 18% isocratic water preparation according to Table 2.

TABLE 5
Chromatography of the Impurities Marker Solution using
isocratic HILIC with 18% water and 346 nM Detection

| | Time (Minutes) | Absorbance (AU) |
|---|---|---|
| Unknown C | approx. 14 | approx. 0.002 |

Table 6 lists the relevant characteristic peaks of isocratic HILIC separation according to the above specifications at a detection wavelength of 346 nm using the 22% isocratic water preparation according to Table 2.

TABLE 6
Chromatography of the Impurities Marker Solution using
isocratic HILIC with 22% water and 346 nM Detection

| | Time (Minutes) | Absorbance (AU) |
|---|---|---|
| Unknown C | approx. 8.5 | approx. 0.002 |

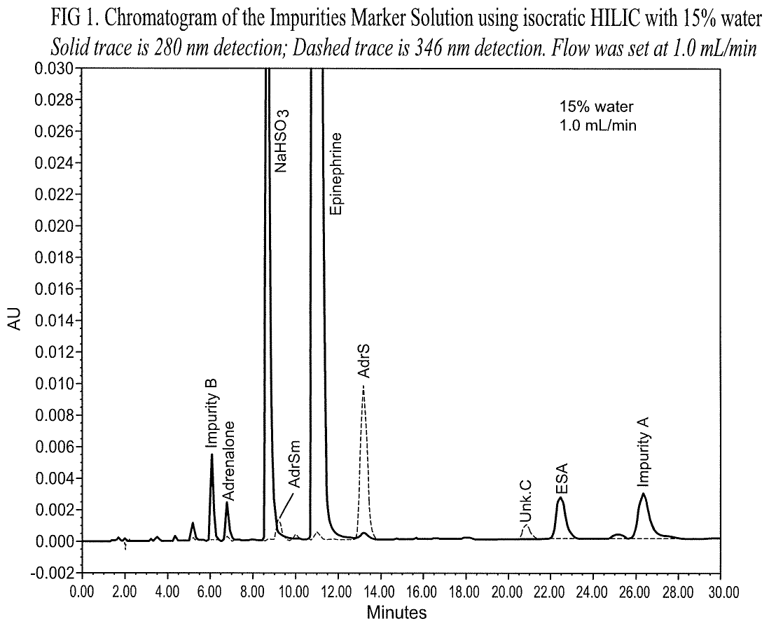

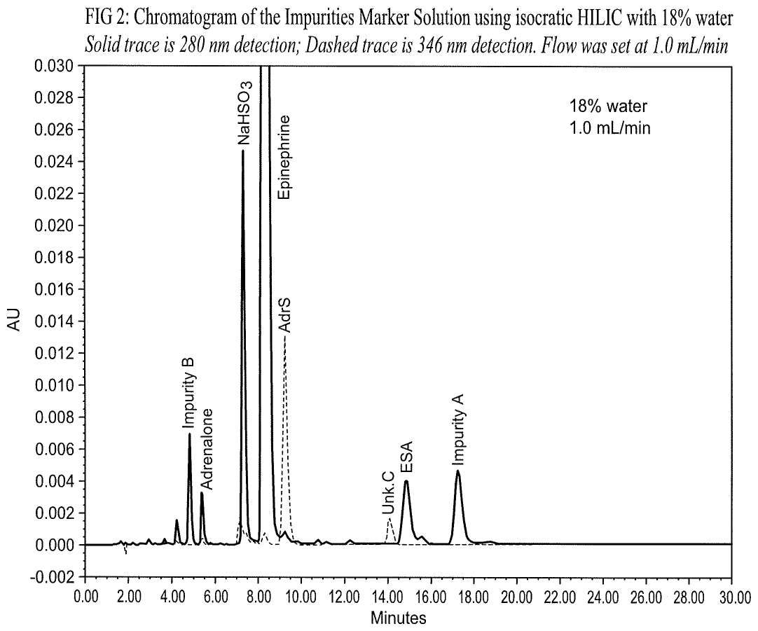

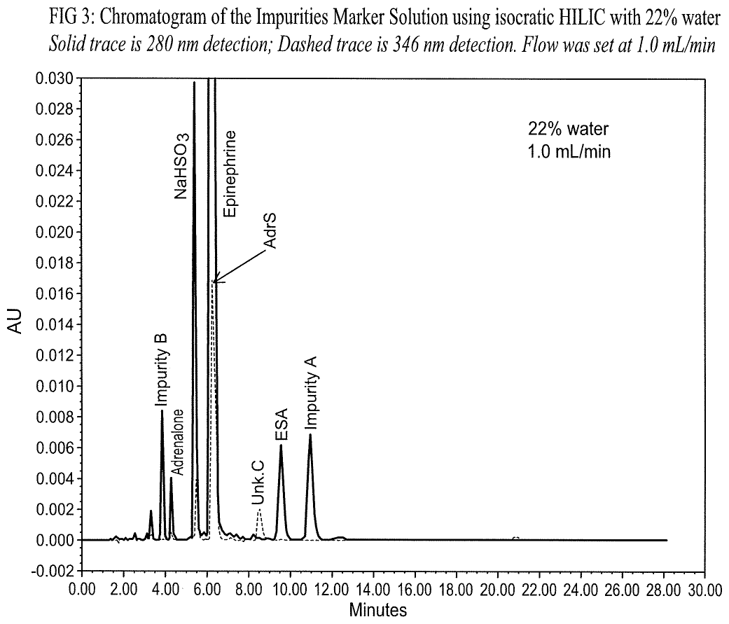

The chromatography outputs of the Impurities Marker Solution using isocratic HILIC as described is also shown in FIG. 1 (15% water), FIG. 2 (18% water), and FIG. 3 (22% water).

Unknown C may be characterized using a gradient HILIC method. An Epinephrine HILIC Impurities Marker Solution (EpiHILIC IMS) which comprises Unknown C may be analyzed using the conditions listed in Table 7 and 8.

Table 7 lists the compositions of the mobile phases that may be used for gradient HILIC separations.

TABLE 7
Composition of Gradient Mobile Phases

| | |
|---|---|
| Mobile Phase A | 100 mL Ammonium Formate Buffer |
| | 900 mL Acetonitrile |
| Mobile Phase A | 100 mL Ammonium Formate Buffer |
| | 400 mL Water |
| | 500 mL Acetonitrile |

Table 8 lists the solvent program for the gradient HILIC method.

TABLE 8
Solvent program for the gradient HILIC method

Mobile Phase Composition

| Time (min) | Flow (mL/min) | Water % | A % | B % |
|---|---|---|---|---|
| Initial | 1.0 | 15.2 | 87 | 13 |
| 12 | 1.0 | 15.2 | 87 | 13 |
| 22 | 1.0 | 22 | 70 | 30 |
| 25 | 1.0 | 22 | 70 | 30 |
| 28 | 1.0 | 15.2 | 87 | 13 |
| 35 | 1.0 | 15.2 | 87 | 13 |

The chromatographic conditions for the gradient HILIC are the same as those listed in Table 3.

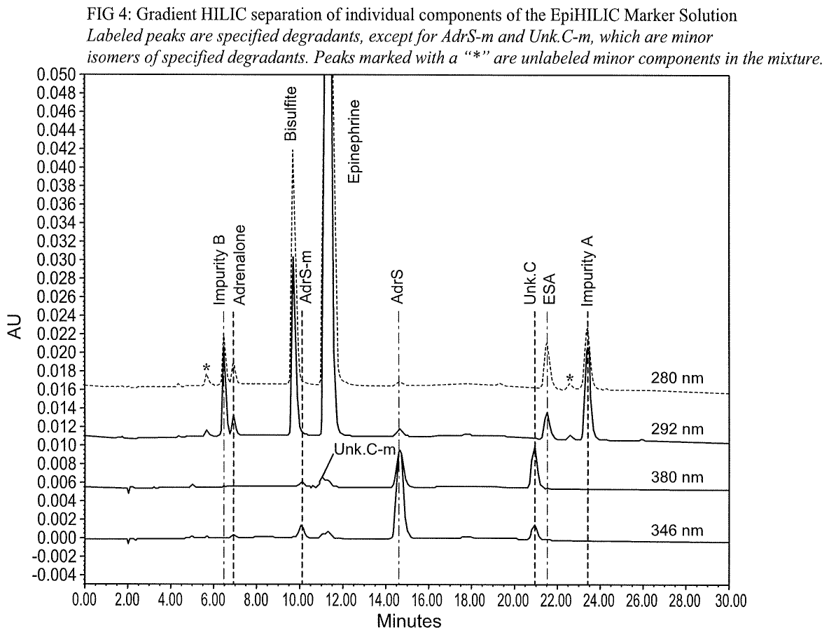

The chromatography outputs of the Impurities Marker Solution using gradient HILIC according to the above specifications at detection wavelengths of 280 nm, 292 nm, 346 nm, and 380 nm are shown in FIG. 4.

According to some aspects, the concentration Impurity A, Impurity B, and Unknown C together in the composition after a certain period of shelf life is no more than about 5%, preferably no more than about 4%, more preferably no more than about 3%, more preferably no more than about 2%, more preferably no more than about 1%, more preferably no more than about 0.5%, more preferably no more than about 0.4%, more preferably no more than about 0.3%, more preferably no more than about 0.2%, and most preferably no more than about 0.1%

According to some aspects, the concentration of Impurity A in the composition after a certain period of shelf life may be no more than about 2%, preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to some aspects, the concentration of Impurity B in the composition after a certain period of shelf life may be no more than about 2%, preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to aspects, the concentration of Unknown C in the composition after a certain period of shelf life may be no more than about 2%, preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to some aspects, the composition may have an extended shelf life. As used throughout this application, the term “shelf life” refers to the length of time that a product may be stored without becoming unfit for medical use. Examples of compositions which are unfit for medical use include, but are not limited to, compositions with unacceptably high impurity levels and/or the presence of physical changes described herein, such as, but not limited to, color change and/or the presence of insoluble particles.

The period of shelf life of the composition may be 1 month, preferably 2 months, more preferably 3 months, more preferably 4 months, more preferably 5 months, more preferably 6 months, more preferably 7 months, more preferably 8 months, more preferably 9 months, more preferably 10 months, more preferably 11 months, more 12 months, preferably 13 months, more preferably 14 months, more preferably 15 months, more preferably 16 months, more preferably 17 months, more preferably 18 months, more preferably 19 months, more preferably 20 months, more preferably 21 months, more preferably 22 months, more preferably 23 months, more preferably 24 months, more preferably 25 months, more preferably 26 months, more preferably 27 months, more preferably 28 months, more preferably 29 months, more preferably 30 months, more preferably 31 months, more preferably 32 months, more preferably 33 months, more preferably 34 months, more preferably 35 months, and most preferably 36 months. According to some aspects, the period of shelf life may vary based on product presentation.

According to some aspects, shelf life may be determined by measuring certain characteristics of the composition that may indicate that the composition is unfit for medical use. According to some aspects, shelf life may be determined by measuring the concentration of impurities in the composition after storage at 25° C. and 60% relative humidity. According to some aspects, shelf life may be determined by assaying the amount of the active ingredient still present in its unaltered form. According to some aspects, shelf life may be determined by measuring the concentration of impurities in the composition after storage at 37° C. and 65% relative humidity.

According to some aspects, shelf life may be determined by measuring the concentration of impurities in the composition using the guidelines as outlined in the ICH Harmonised Tripartite Guideline: Stability Testing of New Drug Substances and Products Q1A(R2), dated Feb. 6, 2003, the disclosure of which is incorporated by reference herein in its entirety.

For example, shelf life may be determined for long term, accelerated, and, where appropriate, intermediate storage conditions by measuring the concentration of impurities after storage in the following conditions, wherein the composition is packaged in a container closure system that is the same as or simulates the packaging proposed for storage and distribution.

| Study | Storage condition |
|---|---|
| Long term | 25° C. ± 2° C./60% RH ± 5% RH |
| | or |
| | 30° C. ± 2° C./65% RH ± 5% RH |
| Intermediate | 30° C. ± 2° C./65% RH ± 5% RH |
| Accelerated | 40° C. ± 2° C./75% RH ± 5% RH |

According to some aspect, shelf life may be attributed at least in part to the concentration of impurities in the composition such that the reduction of impurity concentration and/or rate of impurity formation lengthens the composition's shelf life.

According to some aspect, the composition may have an initial pH. The initial pH of the composition may be such that the rate of component degradation is minimized. For example, the initial pH of the composition may be such that the rate of formation of D-epinephrine is minimized after a certain period of shelf life. According to some aspect, the initial pH range of the composition may be in the range of about 3.5 and 4.5, optionally in the range of about 3.6 and 4.5, optionally in the range of about 3.7 and 4.4, optionally in the range of about 3.8 and 4.3, optionally in the range of about 3.9 and 4.2, optionally in the range of about 4 and 4.2, optionally about 4.1.

According to some aspects, the initial pH may be such that the composition conforms to industry requirements, such as the limit on total acidity imposed by the USP 37-NF 32, S2, monograph.

As described herein, the composition may comprise a buffer system so as to resist significant pH change after a certain period of shelf life. According to some aspects, the pH of the composition after a certain period of shelf life may be no more than about ±1 of the initial pH, optionally no more than about ±0.9 of the initial pH, optionally no more than about ±0.8 of the initial pH, optionally no more than about ±0.7 of the initial pH, optionally no more than about ±0.6 of the initial pH, optionally no more than about ±0.5 of the initial pH, optionally no more than about ±0.4 of the initial pH, optionally no more than about ±0.3 of the initial pH, optionally no more than about ±0.2 of the initial pH, optionally no more than about ±0.1 of the initial pH.

According to some aspects, the pH range after a certain period of shelf life of the composition may be such that the degradation of components of the composition is reduced. For example, the pH range after a certain period of shelf life of the composition may be such that the formation of D-epinephrine is reduced. According to some aspects, the pH range after a certain period of shelf life of the composition may be such that the amount of D-epinephrine present in the composition is in a preferred range described herein.

According to some aspects, the pH range after a certain period of shelf life may be such that the degradation of the antioxidant is reduced. For example, for compositions containing sulfites, the conversion of bisulfite to sulfer dioxide may be reduced. The reduction of the conversion of bisulfite to sulfer dioxide may at least in part lead to a higher proportion of antioxidant present in the composition after a certain period of shelf life compared to compositions outside of the preferred pH range.

According to some aspects, the pH of the composition after a certain period of shelf life may be such that the composition conforms to the limit on total acidity imposed by the USP 37-NF 32, S2, monograph.

According to some aspects, the composition may be provided as a single-dose or multi-dose formulation. According to some aspects, the composition may be contained in vials. According to some aspects, the vials may comprise clear glass, amber glass, or plastic. According to some aspects, the vials may be in the range of about 0.1 to 500 mL in volume, preferably in the range of about 0.5 to 250 mL, more preferably in the range of about 1 to 100 mL, and most preferably in the range of about 10 to 50 mL. According to some aspects, the composition may exist in a 1 mL vial. According to some aspects, the composition may exist in a 10 mL vial. According to some aspects, the composition may exist in a 30 mL vial. According to some aspects, the 1 mL vial may be a single-dose formulation. According to some aspects, the 10 mL vial may be a single-dose formulation. According to some aspects, the 10 mL vial may be a multi-dose formulation. According to some aspects, the 30 mL vial may be a multi-dose formulation. According to some aspects, the same vial may be used for multiple applications of the composition for up to about 10 days after initial use, preferably up to about 15 days, more preferably up to about 30 days, more preferably up to about 45 days, and most preferably up to about 60 days. In some embodiments, the composition may be lyophilized.

According to some aspects, the composition may be contained in an autoinjector.

According to some aspects, the composition may be contained in a pre-filled syringe. In some examples, the pre-filled syringe may comprise a plunger stopper, a barrel, a needle adapter, a needle hub, and a needle suitable for injection. According to some aspects, the pre-filled syringe may comprise glass, rubber, or a combination thereof, such as a glass barrel and a rubber plunger stopper.

The pre-filled syringe may have a labeled size. As used herein, the term “labeled size” refers to the pre-filled syringe's pharmaceutical composition holding capacity and is equal to the volume of a pharmaceutical composition containable in the pre-filled syringe. The pre-filled syringe may have a labeled size of 0.5 mL, optionally 1 mL, optionally 1.5 mL, optionally 2 mL, optionally 2.5 mL, optionally 3 mL, optionally 3.5 mL, optionally 4 mL, optionally 4.5 mL, optionally 5 mL, optionally 5.5 mL, optionally 6 mL, optionally 6.5 mL, optionally 7 mL, optionally 7.5 mL, optionally 8 mL, optionally 8.5 mL, optionally 9 mL, optionally 9.5 mL, optionally 10 mL, optionally 10.1 mL, optionally 10.2 mL, optionally 10.3 mL, optionally 10.4 mL, and optionally 10.5 mL.

According to some aspects, the composition is sterile. As used herein, “sterile” refers to meeting sterility requirements for injection into the human body. According to some aspects, sterility may require passing results when measured via the membrane filtration method and/or direct inoculation method as set forth in USP chapter <71>.

The present disclosure is also directed to a method of providing a composition as described herein. The method may comprise a preparation step, a filtration step, and/or a containment step. It should be understood that as used herein, the term “step” is not particularly limiting. For example, each step as described herein may be a discrete step such that steps are performed sequentially upon completion of a preceding step. Alternatively, at least a portion of the method may be continuous, or the steps may be performed out of the order stated.

The method may comprise a preparation step which includes preparing a batch solution. According to some aspects, preparing the batch solution comprises providing a solvent as described herein having an initial dissolved oxygen content and sparging the solvent with a first inert gas until the solvent has a reduced dissolved oxygen content that is lower than the initial dissolved oxygen content. In one non-limiting example, the solvent, such as WFI, may be provided in a first manufacturing tank and purged/sparged with a first inert gas until the reduced dissolved oxygen content obtained. According to some aspects, the dissolved oxygen content may be measured by a WFI USP/EP 02 probe.

Examples of inert gases according to the present disclosure include, but are not limited to, helium, neon, argon, krypton, xenon, radon, nitrogen, carbon dioxide, and combinations thereof.

According to some aspects, the reduced dissolved oxygen content may be no more than about 1.0 ppm, optionally no more than about 0.9 ppm, optionally no more than about 0.8 ppm, optionally no more than about 0.7 ppm, optionally no more than about 0.6 ppm, optionally no more than about 0.5 ppm, optionally no more than about 0.4 ppm, optionally no more than about 0.3 ppm, optionally no more than about 0.2 ppm, and optionally no more than about 0.1 ppm.

According to some aspects, the preparation step may comprise providing and/or maintaining the solvent at an acceptable temperature, such as a temperature standard to continuous circulation processes. In one non-limiting example, the solvent may be provided and/or maintained at a temperature of from about 20 to 25° C. for at least a portion of the preparation step.

The preparation step may comprise one or more qualitative and/or quantitative measurements. For example, the preparation step may comprise measuring the dissolved oxygen level of the solvent before, during, and/or after sparging as described herein. According to some aspects, the method requires obtaining a satisfactory result from the one or more qualitative and/or quantitative measurements before proceeding to the next step of the process.

According to some aspects, the preparation step may comprise a dissolving step wherein one or more of an active agent, an antioxidant, a pH adjusting agent, a tonicity adjusting agent, a chelating agent, and a buffer system as described herein is dissolved in the solvent. As used herein, the term “dissolve” means to pass into solution, such as by mixing, stirring, or the like. It should be understood that each of the components as described herein may individually be dissolved simultaneously or sequentially with another component. Additionally or alternatively, one or more of the components as described herein may be combined with one or more other components prior to being dissolved in the solvent.

According to some aspects, the solvent may be sparged with the first inert gas before, during, and/or after each of the one or more of the components or combinations thereof is dissolved in the solvent. Alternatively, the solvent may be continuously sparged with the first inert gas during the preparation step such that there is no break in sparging.

According to some aspects, the preparation step may comprise combining the solvent with additional solvent after one or more of the components described herein have been dissolved in order to provide final concentrations for one or more of the components as described herein. The additional solvent may be the same as or different from the solvent. For example, the preparation step may comprise adding additional WFI to the solvent, wherein the additional WFI has been sparged with an inert gas as described herein. According to some aspects, the solvent may be sparged with the first inert gas before, during, or after the additional solvent is combined therewith.

According to some aspects, the batch solution has a final dissolved oxygen content at the end of the preparation step. As used herein, a “final dissolved oxygen content” is the dissolved oxygen content of the batch solution before the batch solution is utilized in a next step in the method, such as the filtration step and/or the containment step.

According to some aspects, the final dissolved oxygen content may be no more than no more than about 1.9 ppm, optionally no more than about 1.8 ppm, optionally no more than about 1.7 ppm, optionally no more than about 1.6 ppm, optionally no more than about 1.5 ppm, optionally no more than about 1.4 ppm, optionally no more than about 1.3 ppm, optionally no more than about 1.2 ppm, optionally no more than about 1.1 ppm, optionally no more than about 1.0 ppm, optionally no more than about 0.9 ppm, optionally no more than about 0.8 ppm, optionally no more than about 0.7 ppm, optionally no more than about 0.6 ppm, optionally no more than about 0.5 ppm, optionally no more than about 0.4 ppm, optionally no more than about 0.3 ppm, optionally no more than about 0.2 ppm, and optionally no more than about 0.1 ppm.

In one non-limiting example of a method according to the present disclosure, the preparation step comprises providing WFI as a solvent, cooling the WFI to about 20 to 25° C., and sparging the WFI with nitrogen until the WFI has a reduced dissolved oxygen content as described herein. The preparation step may then comprise sequentially dissolving a tonicity adjusting agent, buffer components of a buffer system, a chelating agent, and an antioxidant in the solvent, wherein the solvent is continuously sparged as the components are dissolved therein, and wherein the solvent is mixed after the addition of each component. The preparation step may further comprise separately combining a pH adjusting agent with an active agent and mixing to provide an active agent solution. The active agent solution may then be dissolved in the solvent while the solvent is continuously sparged. The preparation step may comprise adding additional WFI to the solvent to bring the batch solution to a final volume, where the additional WFI has been sparged with nitrogen. In this example, the resulting batch solution may have a final dissolved oxygen content as described herein.

The method may comprise a filtration step wherein at least a portion of the batch solution from the preparation step is passed through one or more filters. Each of the one or more filters may independently be the same as or different from another filter used in the method.

According to some aspects, the one or more filters may be sufficient to provide a sterile composition as described herein. For example, each of the one or more filters may independently be a sterilizing-grade filter comprising a membrane having an average pore size of between about 0.1 and 1 μm, optionally between about 0.2 and 1 μm, optionally between about 0.2 and 0.45 μm, optionally about 0.45 μm, optionally about 0.2 μm, and optionally about 0.22 μm. According to some aspects, each of the one or more filters may independently comprise a membrane having pores rated for mycoplasma and/or other small microbes. Example materials useful for the one or more filters include, but are not limited to, regenerated cellulose, polymers such as polyethersulfone (PES), polyvinylidene fluoride (PVDF), and polytetrafluoroethylene (PTFE), and combinations thereof. Non-limiting examples of sterilizing-grade filters useful according to the present disclosure include Merck's OptiScale® 25 Milligard PES 1.2/0.45 μm NB and Merck's Millipore Express® SHF 2.0 Capsule Filter. According to some aspects, the method may comprise passing the solvent having the active agent and the pH adjusting agent solubilized therein through a filter combination comprising Merck's OptiScale® 25 Milligard PES 1.2/0.45 μm NB and Merck's Millipore Express® SHF 2.0 Capsule Filter.

According to some aspects, the method may comprise a containment step wherein at least a portion of the batch solution is provided in a container as described herein, such as in a pre-filled syringe or a component thereof (e.g., in a barrel of a pre-filled syringe), in order to provide a pharmaceutical composition in a container as described herein. The containment step may further comprising sealing the container (e.g., via a plunger stopper). It should be understood that the batch solution may be sterile as described herein if the containment step follows a filtration step as described herein. Containing at least a portion of the batch solution in the container and/or component thereof may be accomplished using any machinery known in the art capable of filling a pre-filled syringe and/or component thereof with a composition as described herein. According to some aspects, the batch solution may be sparged with a second inert gas before, during, and/or after any portion of the containment step, wherein the second inert gas is the same as or different from the first inert gas. For example, the batch solution may be sparged with the second inert gas as it is provided in the barrel of a pre-filled syringe and/or as the barrel of the pre-filled syringe is sealed with a plunger stopper. According to some aspects, the batch solution may be sparged with the second inert gas continuously throughout the containment step.

The method may further comprise providing the container containing the pharmaceutical solution in a storage receptacle with protection from light.

The present invention also provides for methods of treating or reducing the symptoms associated with a medical condition, comprising administering to a subject in need thereof the pharmaceutical composition of the present invention.

Examples of conditions to be treated comprise bronchospasm, sensitivity reactions, cardiac arrhythmias, GI and renal hemorrhage, superficial bleeding, premature labor, hypoglycemia, and cardiogenic, hemorrhagic, and traumatic shock. In some embodiments, the present invention provides for methods for reducing the symptoms associated with allergic reactions (Type 1), including anaphylaxis, and/or for the induction and maintenance of mydriasis during intraocular surgery.

Examples of methods of administration comprise subcutaneous, intracameral, intravenous, and intramuscular injection, infusion, intra-arterial administration, intracardiac injection, endotracheal administration, intraosseous administration, oral inhalation, topical administration, and as ophthalmic irrigation.

Unless otherwise noted, all concentrations herein are expressed as weight/volume percent (w/v %) of the composition.

The present invention is further described by way of the following non-limiting Examples that are given for illustrative purposes only.

### EXAMPLES

### Example I: Preparation of Pharmaceutical Composition in Pre-Filled Syringe

A sterile pharmaceutical composition having the formulation shown in Table 8 was prepared.

TABLE 8
Epinephrine Formulation

| Component | Concentration (mg/mL) |
|---|---|
| Epinephrine | 0.1 |
| Sodium metabisulfite | 0.0457 |
| Sodium chloride | 9 |
| Tartaric acid | 0.225 |
| Disodium edetate, dihydrate | 0.02 |
| Hydrochloric acid 4% solution | 0.606 |
| Sodium hydroxide | 0.1 |
| Water for injection | Q.S. |

To prepare the formulation, an 80% batch size of WFI was collected in a bulk manufacturing tank and cooled to 20 to 25° C. The WFI was then sparged until the dissolved oxygen content (DO) was less than 0.5 ppm. Then, sodium chloride was added to the bulk manufacturing tank, and the solution was mixed until the sodium chloride was dissolved. Tartaric acid was then added to the bulk manufacturing tank, and the solution was mixed until the tartaric acid was dissolved. Then, sodium hydroxide was added to the bulk manufacturing tank, and the solution was mixed until the sodium hydroxide was dissolved. Then, disodium edetate dihydrate was added to the bulk manufacturing tank, and the solution was mixed until the disodium edetate dihydrate was dissolved. Sodium metabisulfite was then added to the bulk manufacturing tank, and the solution was mixed until the sodium metabisulfite was dissolved. The solution in the bulk manufacturing tank was continuously sparged with nitrogen throughout the process.

The epinephrine was provided in a separate epinephrine dispensing bottle, and a Teflon stir bar was added. The hydrochloric acid 4% solution was then added to the epinephrine dispensing bottle and was mixed with a magnetic stirrer to provide an epinephrine solution. Once combined, the epinephrine solution was added to the solution in the bulk manufacturing tank while the solution in the bulk manufacturing tank was continuously sparged with nitrogen.

Finally, nitrogen sparged WFI was added to adjust the final weight of the solution in the bulk manufacturing tank to provide a batch solution. The final DO of the batch solution was less than 0.9 ppm.

The batch solution was then filtered through a 0.22μ sterile PVDF filter and filled in 10 mL glass syringe barrels with 10.2 ml fill volume. Plunger stoppers were placed into the syringe barrels under nitrogen flushing and sealed, labeled, and stored in carton to protect from light.

### Example II: Stability of Pharmaceutical Composition

The stability of the pharmaceutical compositions prepared according to Example I was compared with a comparative product, the 1 mg/10 mL (0.1 mg/mL) epinephrine injection distributed by Hospira, Inc. The comparative product is a clear and colorless solution available in glass vials. Each vial is co-packaged with an injector, which together make a single dose ABBOJECT syringe. The formulation of the comparative product is shown in Table 9.

TABLE 9
Comparative Formulation

| Component | Concentration (mg/mL) |
|---|---|
| Epinephrine | 0.1 |
| Sodium metabisulfite | 0.46 |
| Sodium chloride | 8.16 |
| Citric acid, anhydrous | 2.13 |
| Sodium citrate, dihydrate | 0.41 |
| Citric acid, anhydrous | Q.S. to pH |
| Sodium citrate, dihydrate | Q.S. to pH |
| Water for injection | Q.S. |

Stability data was recorded for the pharmaceutical composition of Example I and for the comparative product after three months and six months at accelerated storage conditions (40° C./75% RH). The results of the study are shown in Tables 10 and 11, respectively.

TABLE 10
Stability Data after Three Months
in Accelerated Storage Conditions

| | Comparative | Inventive |
|---|---|---|
| pH | 3.19 | 4.09 |
| Osmolality (mOSm/kg) | 300 | 291 |
| L- Epinephrine | 99.1% (w/v) | 96.4% (w/v) |
| D-Epinephrine | 7.58% (w/v) | 1.4% (w/v) |
| Adrenolone | 0.04% (w/v) | 0.01% (w/v) |
| ESA | 6.85% (w/v) | 4.59% (w/v) |
| Unk B | 0.12% (w/v) | 0.18% (w/v) |
| Unk A | 0.13% (w/v) | 0.20% (w/v) |
| AdrS | 0.04% (w/v) | ND |
| Unk C | 0.13% (w/v) | 0.03% (w/v) |
| Individual unspecified impurity | 2.22% (w/v) | 0.01% (w/v) |
| Total Impurities | 10.05% (w/v) | 5.04% (w/v) |

TABLE 11
Stability Data after Six Months in Accelerated Storage Conditions

| | Comparative | Inventive |
|---|---|---|
| pH | 3.15 | 4.13 |
| Osmolality (mOSm/kg) | 294 | 300 |
| L- Epinephrine | 94.0% (w/v) | 90.7% (w/v) |
| D-Epinephrine | 13.17% (w/v) | 2.2% (w/v) |
| Adrenolone | 0.05% (w/v) | 0.01% (w/v) |
| ESA | 11.57% (w/v) | 7.07% (w/v) |
| Unk B | 0.19% (w/v) | 0.43% (w/v) |
| Unk A | 0.09% (w/v) | 0.57% (w/v) |
| AdrS | 0.05% (w/v) | 0.01% (w/v) |
| Unk C | 0.13% (w/v) | 0.02% (w/v) |
| Individual unspecified impurity | 1.99% (w/v) | 0.20% (w/v) |
| Total Impurities | 14.08% (w/v) | 10.62% (w/v) |

Based on the above, it was determined that the process of preparing the inventive pharmaceutical composition imparted significant effects on the final composition compared with a commercial product. Notably, the inventive pharmaceutical composition showed markedly enhanced stability compared with the commercial product.

### Example III: Pharmaceutical Composition Stability Profile

Various parameters of the pharmaceutical composition prepared according to Example I were studied under long-term (25° C./60% RH), intermediate (30° C./65% RH), and accelerated (40° C./75% RH) storage conditions. The results are shown in Table 12.

TABLE 12
Stability Data at Different Storage Conditions

| Condition: | | | 25° C./60% RH | | 30° C./65% RH | | 40° C./75% RH | |
|---|---|---|---|---|---|---|---|---|
| Test | Specification | Initial | 3M | 6M | 3M | 6M | 3M | 6M |
| Description | Clear | CCS | CCS | CCS | CCS | CCS | CCS | CCS |
| | Colorless | | | | | | | |
| | Solution | | | | | | | |
| pH | ND | 4.11 | 4.10 | 4.13 | 4.10 | 4.11 | 4.09 | 4.13 |
| Osmolality | 260-320 | 286 | 291 | 301 | 287 | 299 | 291 | 300 |
| (mOSm/kg) | mOSm/kg | | | | | | | |
| % Assay | 90% to 115% | 100.6 | 100.5 | 97.5 | 98.3 | 96.6 | 97.8 | 92.9 |
| Sodium | 10% to 110% | 98.2 | 97.79 | 92.65 | 93.62 | 87.15 | 82.13 | 68.22 |
| metabisulfite | | | | | | | | |
| Content (%) * | | | | | | | | |
| EDTA content | 90% to 110% | 99.2 | 100.1 | 101.4 | 95.8 | 101.2 | 89.1 | 101.4 |
| Total Acidity | NLT 25 mL | Complies | Complies | Complies | Complies | Complies | Complies | Complies |
| Color And Clarity | Meets USP | Complies | Complies | Complies | Complies | Complies | Complies | Complies |
| | Requirement | | | | | | | |
| Isomer Content | | | | | | | | |
| L-Epinephrine | 90% to 115% | 100.1 | 100 | 97 | 97.7 | 95.9 | 96.4 | 90.7 |
| (% w/v) | | | | | | | | |
| D-Epinephrine | NMT 5% | 0.5 | 0.5 | 0.5 | 0.6 | 0.7 | 1.4 | 2.2 |
| (% w/v) | | | | | | | | |
| Related Substance | | | | | | | | |
| Adrenolone | NMT 0.5% | ND | ND | 0.00 | 0.00 | 0.01 | 0.01 | 0.01 |
| (% w/v) | | | | | | | | |
| ESA (% w/v) | NMT 13.5% | ND | 0.76 | 1.16 | 1.31 | 2.26 | 4.59 | 7.07 |
| Imp B (% w/v) | NMT 1.0% | ND | 0.04 | 0.09 | 0.09 | 0.16 | 0.18 | 0.43 |
| Imp A (% w/v) | NMT 1.0% | ND | 0.05 | 0.10 | 0.07 | 0.22 | 0.20 | 0.57 |
| AdrS (% w/v) | NMT 0.2% | ND | ND | ND | ND | 0.01 | ND | 0.01 |
| Unk C (% w/v) | NMT 1.5% | ND | ND | 0.00 | 0.01 | 0.01 | 0.03 | 0.02 |
| Individual | NMT 0.5% | ND | 0.01 | ND | 0.01 | ND | 0.01 | 0.20 |
| unspecified | | | | | | | | |
| impurity (% w/v) | | | | | | | | |
| Total Impurities | NMT 17% | 0.46 | 0.84 | 1.82 | 1.49 | 3.36 | 5.04 | 10.62 |
| (% w/v) | | | | | | | | |

We claim:

1. A method of making a pharmaceutical composition in a pre-filled syringe comprising:
(a) preparing a batch solution, wherein preparing the batch solution comprises:
providing a solvent having an initial dissolved oxygen content,
sparging the solvent with a first inert gas until the solvent has a reduced dissolved oxygen content that is lower than the initial dissolved oxygen content, wherein the reduced dissolved oxygen content is less than about 0.5 ppm, and
combining the solvent with an active agent and an antioxidant, wherein the solvent is continuously sparged with the first inert gas during combination with the active agent and the antioxidant, and
(b) introducing at least a portion of the batch solution of (a) into a syringe to provide the pharmaceutical composition in the pre-filled syringe,
wherein the batch solution of (a) is sparged with a second inert gas when provided in the pre-filled syringe, and
wherein the second inert gas is the same as or different from the first inert gas,
wherein the pharmaceutical composition in the pre-filled syringe has a concentration of total impurities of the active agent of no more than about 5.5% (w/v) after three months of storage in accelerated storage conditions.

2. The method of claim 1, wherein the active agent comprises epinephrine.

3. The method of claim 2, wherein the antioxidant comprises sodium metabisulfite and the pharmaceutical composition in the prefilled syringe has a concentration of sodium metabisulfite of from about 0.001 to about 0.2 mg/mL.

4. The method of claim 2, wherein the pharmaceutical composition in the prefilled syringe has a concentration of epinephrine of from about 0.01 to about 2 mg/mL.

5. The method of claim 1, wherein the first inert gas comprises nitrogen.

6. The method of claim 1, wherein the solution of (a) is passed through a filter prior to being provided in the pre-filled syringe such that the pharmaceutical composition in the pre-filled syringe is sterile.

7. The method of claim 6, wherein the filter comprise pores with an average pore size of about 0.22 μm.

8. The method of claim 1, wherein preparing the solution of (a) further comprises combining the solvent with one or more of a tonicity adjusting agent, a buffer system, a chelating agent, and a pH adjusting agent.

9. The method of claim 8, wherein the tonicity adjusting agent comprises sodium chloride, the buffer system comprises tartaric acid and sodium hydroxide, the chelating agent comprises EDTA, and the pH adjusting agent comprises hydrochloric acid.

10. The method of claim 1, wherein the solvent comprises water for injection (WFI).

11. The method of claim 1, wherein preparing the solution of (a) further comprises combining the solvent with additional solvent after combination with the active agent and/or the antioxidant, and wherein the solution of (a) has a final dissolved oxygen content of no more than about 0.9 ppm.

12. The method of claim 2, wherein the pharmaceutical composition in the pre-filled syringe has a concentration of D-epinephrine of no more than about 5% (w/v) after three months of storage in accelerated storage conditions.

13. The method of claim 12, wherein the concentration of D-epinephrine after three months of storage in accelerated storage conditions is no more than about 1.4% (w/v).

14. The method of claim 2, wherein the pharmaceutical composition in the pre-filled syringe has a concentration of D-epinephrine of no more than about 5% (w/v) after six months of storage in accelerated storage conditions.

15. The method of claim 14, wherein the concentration of D-epinephrine after six months of storage in accelerated storage conditions is no more than about 2.2% (w/v).

16. A pharmaceutical composition provided in a pre-filled syringe prepared by a method comprising:
(a) preparing a batch solution, wherein preparing the batch solution comprises:
providing a solvent having an initial dissolved oxygen content,
sparging the solvent with a first inert gas until the solvent has a reduced dissolved oxygen content that is lower than the initial dissolved oxygen content, wherein the reduced dissolved oxygen content is less than about 0.5 ppm, and
combining the solvent with an active agent and an antioxidant, wherein the solvent is continuously sparged with the first inert gas during combination with the active agent and the antioxidant, and
(b) introducing at least a portion of the batch solution of (a) into a syringe to provide the pharmaceutical composition in the pre-filled syringe,
wherein the batch solution of (a) is sparged with a second inert gas when provided in the pre-filled syringe, and
wherein the second inert gas is the same as or different from the first inert gas,
wherein the pharmaceutical composition in the pre-filled syringe has a concentration of total impurities of the active agent of no more than about 5.5% (w/v) after three months of storage in accelerated storage conditions.

17. The pharmaceutical composition of claim 16, wherein the active agent comprises epinephrine, and wherein the pharmaceutical composition in the pre-filled syringe has a concentration of D-epinephrine of no more than about 5% (w/v) after three months of storage in accelerated storage conditions.

18. The pharmaceutical composition of claim 17, wherein the concentration of D-epinephrine after three months of storage in accelerated storage conditions is no more than about 1.4% (w/v).

19. The pharmaceutical composition of claim 16, wherein the active agent comprises epinephrine, and wherein the pharmaceutical composition in the pre-filled syringe has a concentration of D-epinephrine of no more than about 5% (w/v) after six months of storage in accelerated storage conditions.

20. The pharmaceutical composition of claim 19, wherein the concentration of D-epinephrine after six months of storage in accelerated storage conditions is no more than about 2.2% (w/v).
